(19) United States
(12) Patent Application Publication
(10) Pub. No.: US 2006/0084159 A1
(43) Pub. Date: Apr. 20, 2006

(54) Method for manufacturing of three dimensional composite surfaces for microarrays

(75) Inventor: Vladimir Trubetskoy, Middleton, WI (US)

Correspondence Address: Medlen & Carroll, LLP, Suite 350, 101 Howard Street, San Francisco, CA, 94105, US

(73) Assignee: Quintessence Biosciences, Inc., Madison, WI (US)

(21) Appl. No.: 11/214,092

(22) Filed: Aug. 29, 2005

Related U.S. Application Data
(60) Provisional application No. 60/605,316, filed on Aug. 27, 2004.
(60) Provisional application No. 60/699,150, filed on Jul. 14, 2005.

(51) Int. Cl. C12N 11/16

(52) U.S. Cl. 435/174

(57) ABSTRACT

The present invention is directed toward the manufacturing of three-dimensional polymeric coatings for molecule (e.g., protein) immobilization on a solid surface (e.g., surface of a glass slide, microwell plate, etc.). Such surfaces find use as microarrays. In some embodiments, the 3D coating are hydrogel-based and comprise a blend of at least two polymers with distinctive roles: 1) inert 3D support; and 2) protein reactive polymer (e.g., primary amine-reactive polymer) which is able to bond to the glass surface and covalently react with proteins. In some embodiments, the 3D coating possesses superabsorbent properties and comprises a crosslinked polyacrylic acid with carboxylic groups activated to react with primary amines of molecules to be arrayed.

The present invention claims priority to U.S. Provisional Patent Application Ser. Nos. 60/605,316, filed Aug. 27, 2004 and 60/699,150, filed Jul. 14, 2005, the disclosures of which are herein incorporated by reference in their entireties.

### FIELD OF THE INVENTION

The present invention is directed toward the manufacturing of three-dimensional polymeric coatings for molecule (e.g., protein) immobilization on a solid surface (e.g., surface of a glass slide, microwell plate, etc.). Such surfaces find use as microarrays. In some embodiments, the 3D coating are hydrogel-based and comprise a blend of at least two polymers with distinctive roles: 1) inert 3D support; and 2) protein reactive polymer (e.g., primary amine-reactive polymer) which is able to bond to the glass surface and covalently react with proteins. In some embodiments, the 3D coating possesses superabsorbent properties and comprises a crosslinked polyacrylic acid with carboxylic groups activated to react with primary amines of molecules to be arrayed.

### BACKGROUND OF THE INVENTION

Three-dimensional (3D) crosslinked hydrophilic polymeric networks are intensively used in many applications including surfaces for nucleic acid- and protein-based microarrays. Analogous to DNA arrays, protein arrays are designed to detect ligands to multiple surface-bound proteins. Fluorescence is a standard detection modality for many microarray applications. The sensitivity of analyte (ligand) detection is directly proportional to its surface density. In this respect the use of 3D polymer networks on glass slide surfaces offers significantly higher binding capacity of the spotted material as compared to regular planar (2D) surfaces (FIG. 1). Fluorescence background depends on optical transparency of a surface material and the presence of fluorescent contaminations (such as the excess of non-bound fluorescently labeled ligand). Both sensitivity and background are critical issues in overall performance of protein microarrays. If the concentration of a ligand is 10× greater but the background is 11× greater, nothing useful is detectable. Additionally, one of the significant problems with 2D planar surfaces is that it is very difficult to get enough biological material to bind to give sufficient assay sensitivity.

### DESCRIPTION OF THE DRAWINGS

FIG. 1 shows a schematic of the concept of polymer interpenetrating network as coating for microarray surfaces.

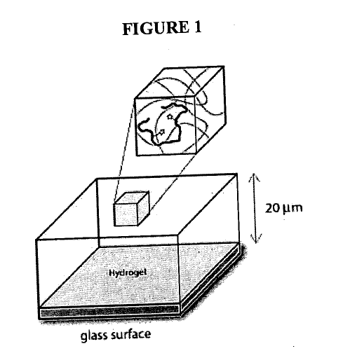

FIG. 2 shows data that demonstrates that the hydrogel-coated surface of one embodiments of the present invention showed significantly higher sensitivity in biotin-BSA/fluorescent streptavidin assay, proving the superiority of 3D hydrogel surface over conventional 2D surfaces.

FIG. 3 shows a comparison of binding assay sensitivity between slides of the present invention (Q-gel) and available commercial slides. Dilutions of biotin-BSA in HEPES-buffered saline were spotted on a coated slide of the present invention and a popular PATH slide (GenTel Biosciences).

FIG. 4 shows detection of fluorescent ligand binding to membrane receptor immobilized on Q-gel surface.

FIG. 5 shows detection of Cytochrome P450 1A2 (baculosomes, Invitrogen) directly immobilized on the Q-gel surface. The immobilized protein was developed using fluorescently-labeled antibodies against CYP1A2.

FIG. 6 shows detection of enzyme activity of Cytochrome P450 immobilized on the Q-gel surface. For this experiment, Q-gel was immobilized on the bottom of 96-well plate.

FIG. 7. shows chemical crosslinking of poly(acrylic acid) with PEG diamine.

FIG. 8. shows binding of Alexa555-streptavidin conjugate to the biotin-BSA covalently attached to the surface of pAA-based hydrogel, FAST and SuperEpoxy slides.

FIG. 9. shows a comparison of background fluorescence readings from various array surfaces.

### SUMMARY OF THE INVENTION

In some embodiments, the present invention relates to the method of manufacturing, for example, glass-bonded 3D composite hydrogel-based polymeric surfaces for microarrays possessing high protein binding capacity and high signal-to-noise (S/N) ratio. The hydrogel is composed of two or more individual polymers with distinctive functions. For example, in some embodiments, one of the constituents forms a structural 3D network, while another carries functional reactive groups that bond the whole composite to the glass surface and bind proteins. The low background is achieved via using a transparent gel layer of low light scattering. High sensitivity is achieved via increased binding capacity of the 3D polymeric network. Thus, an advantage of the invention is that one can bind a lot more material to a surface without producing the corresponding deleterious effects such as background signal, low Z′ factors, loss of accuracy, precision, linearity, robustness, etc.

The present invention also relates to the method of manufacturing a 3D crosslinked polymeric surfaces for microarrays possessing low background fluorescence and high assay sensitivity. The present invention further provides surfaces and microarrays made by such methods. Low background is achieved using, for example, a transparent gel layer of low light scattering. High sensitivity is achieved, for example, by increasing binding capacity of 3D polymeric network.

For example, the present invention provides a composition configured to attach a biological molecule or a collection of biological molecules, wherein the surface comprises an surface layer (e.g., an impermeable layer) and a porous matrix layer, wherein the porous matrix layer comprises cross-linked polymers and has one or more of the following properties: i) superabsorbability; ii) hydrophilic regions; or iii) substantially no fluorescence.

The present invention is not limited by the nature of the biological (or other) molecule that can be attached to the composition. Biological molecules include, but are not limited to proteins, nucleic acids, lipids, carbohydrates, peptides, and synthetic polymers. The compositions of the present invention are particularly useful for the preparation and analysis of protein/peptide arrays (e.g., receptors, immunoglobulins, enzymes, etc.).

In some embodiments, the cross-linked polymers comprise carboxy-containing polymer (e.g., poly(acrylic acid)) activated with water-soluble carbodiimide. In particularly preferred embodiments, the porous matrix layer is formed using a diamine crosslinker of molecular weight greater than 700. In some embodiments, the porous matrix layer is formed using a polyethylene glycol diamine crosslinker. In some preferred embodiments, the polyethylene glycol diamine crosslinker has a molecular weight greater than 700. In some preferred embodiments, the porous matrix layer is lyophilized (e.g., through the use of a cryoprotectant such as trehalose or mannitol, although any suitable cryoprotectant may be used).

The compositions of the present invention may be provided in kits along with other reagents or materials useful in conducting a detection reaction.

The present invention also provides methods for preparing and using such compositions. For example, the present invention provides a method of preparing a highly porous crosslinked polymeric network on a surface, comprising the steps of: a) providing a surface; b) associating a porous matrix layer comprising cross-linked polymers with said surface; wherein said porous matrix layer has one or more properties comprising: i) superabsorbability; ii) hydrophilic regions; and iii) substantially no fluorescence. In preferred embodiments, the cross-linking and surface bonding take place in the same reaction. In some embodiments, the porous matrix layer is associated with the surface by direct casting, dip-coating, or spin-coating.

In some embodiments, the method further comprises the step of attaching a biological molecule to the porous matrix layer (e.g., to form a patterned array). In some embodiments, the method further comprises the step of exposing said patterned array to a sample (e.g., to determine the presence or absence of a molecule interaction of interest between a molecule in the sample and the patterned array via fluorescence detection).

In some embodiments, the present invention provides a composition comprising a surface configured to attach a plurality of biological molecules, said surface comprising a polymeric network having an interpenetrating mesh of two or more non-covalently-linked polymers, the first polymer providing an inert hydrophilic supporting scaffold and the second polymer configured to bond said polymeric network to said surface and said biological molecules. In some embodiments, the first polymer comprises agarose. In some embodiments, the second polymer comprises poly(acrylic acid NHS ester). In some embodiments, the surface further comprises said biological molecules (e.g., nucleic acid, protein, etc.). The composition may be provided as part of a kit. The composition may be used in any desired manner. In some embodiments, the composition is used to screen test samples by exposing the test sample to the composition and monitoring an effect. The test sample may comprise small molecule drugs, biological molecules, etc.

The present invention further provides a method for manufacturing a surface configured to attach a plurality of biological molecules, comprising: contacting a surface with a polymeric network having an interpenetrating mesh of two or more non-covalently-linked polymers, the first polymer providing an inert hydrophilic supporting scaffold and the second polymer configured to bond said polymeric network to said surface and said biological molecules.

Definitions

To facilitate understanding of the invention, a number of terms are defined below.

As used herein, the term “hydrogel” refers to a 3D hydrophilic matrix containing at least 98% water (w/w). Many existing surfaces developed for protein microarray applications are built around hydrogel architecture. Examples include PE's Hydrogel slides (described at the web site for las.perkinelmer.com) and Schott Nexterion H slides (described at the website for www.us.schott.com). However, in existing products the use of the hydrogel concept is not effective for all desired application. For example, load capacity of hydrogel surfaces was reported to be only 1.5-2 times higher than regular 2D planar surfaces (Angenendt et al., Abal. Biochem., 309, 253-260 (2002)). The reason of such inefficiency seems to be small pore size, although an understanding of the mechanism is not necessary to practice the present invention and the present invention is not limited to any particular mechanism of action. PE's Hydrogel is polyacrylamide-based hydrogel. Polyacrylamides form unstable networks at low monomer concentrations thus limiting pore size.

As used herein, the term “label” refers to any particle or molecule that can be used to provide a detectable (preferably quantifiable) effect. In some embodiments, labels utilized in the present invention detect a change in the polarization, position, fluorescent, reflective, scattering or absorptive properties of the reporter molecules.

As used herein, the term “a device configured for the detection of said labels” refers to any device suitable for detection of a signal.

As used herein, the term “molecular recognition element” refers to any molecule or atom capable of detecting a “biological macromolecule” or other desired molecule (e.g., small molecule drug). In some embodiments, molecular recognition elements detect biological molecules present in or attached to the surface. In other embodiments molecular recognition elements detect biological molecules in vitro. In some embodiments, molecular recognition elements are antibodies.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

The term “epitope” as used herein refers to that portion of an antigen that makes contact with a particular immunoglobulin. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

As used herein, the term “polymerization” encompasses any process that results in the conversion of small molecular monomers into larger molecules comprised of repeated units.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

### DETAILED DESCRIPTION OF THE INVENTION

Most methods for manufacturing 3D crosslinked polymer surfaces for microarrays usually involve radical polymerization of acrylic monomers and low molecular weight crosslinkers (Rubina et al., Anal Biochem, 325, 92-106 (2004)). There are several examples of hydrogels being used as porous glass slide surfaces in the patent literature (See e.g., WO 00/33078A1, WO 00/43539A2; U.S. Pat. No. 6,391,937; EP1081163A1; U.S. Pat. No. 5,932,711; U.S. Pat. No. 6,372,813; and U.S. Pat. No. 6,692,914; each of which is herein incorporated by reference in its entirety). Most of these methods involve polymerization of acrylic monomers directly on the surface of a glass slide. An alternative method such as a covalent crosslinking of hydrophilic polymers or use of non-covalent bonding of hydrogel pad to the glass surface have been rarely used.

Recently, in work unrelated to microarray technologies, it was shown that increasing the molecular weight of a crosslinker significantly improves mechanical properties of the 3D network as well as increase pore size (Eiselt et al., Biomaterials, 21, 1921-1927 (2000)). Polymeric poly(ethyleneglycol)-based diamines with molecular weights of 700-3400 have been used as crosslinkers. The crosslinking step is followed by activation of carboxylic groups with water-soluble carbodiimide and N-hydroxysucinimide ester to render gel reactive towards proteins. After activation the gels were extensively washed in water and cryoprotectant solution (10-15% trehalose) and lyophilized. 3D hydrophilic scaffolds of this type have never been used for microarray surface applications.

Another known problem of polymeric microarray surfaces is their high background fluorescence. For example, now popular nitrocellulose membrane-based array substrates have high fluorescence background in 500-600 nm emission range (Fisler, R., American Biotechnology Laboratory, 23-25 (2004)). It seems that this problem is due to non-transparent nature of glass slide coating and caused mainly by light scattering. Agarose gels of low porocity are transparent and, hence, possess very low fluorescence background.

Other hydrogel-based surfaces are already commercially available, such as Packard Hydrogel™-coated slides from Perkin-Elmer Life Science. Such surfaces are modified with proprietary additives that enhance protein binding and preserve protein activity. Xantec Bioanalytics markets biochip surfaces coated with reactive polysaccharide hydrogel. High protein binding membranes prepared of nitrocellulose are another popular technology for manufacturing surfaces for protein microarrays. Such products are produced by Schleicher & Schuell (Keene, N.H.) (FAST slides) and Clinical MicroArrays (Natick, Mass.) (PATH slides). The mode of action of nitrocellulose surfaces (FAST, PATH) is based on the physical adsorption of proteins on the hydrophobic nitrocellulose membrane. A detrimental property of the nitrocellulose is inactivation of some proteins upon adsorption. A problem with FAST slides is their high level of background. While the background problem was solved in part with PATH slides by decreasing thickness, this was done at the expense of adsorption capacity.

Methods of the present invention for preparation of hydrogel-coated slides for protein microarrays, in some embodiments of the present invention, are based on the formation of an interpenetrating network of two or more hydrophilic polymers, each of which performs its own function. One of the polymers should possess the ability to form a 3D hydrophilic and water-insoluble scaffold. The second polymer possesses chemically active groups for protein binding and for bonding the gel to the glass surface. More polymers can be added for more functionality as desired (for example, agarose—support; aldehyde dextran—protein binding; pAA—superabsorbent properties). Preferably, there are no covalent bonds between the two polymeric constituents.

Certain specific embodiments are described below employing agarose as an inert supporting material and NHS ester-activated pAA as a glass bending and protein-binding polymer. The present invention is not limited to these particular examples. Any macroporous neutral hydrophilic three-dimensional matrix can be used as a substitute of agarose. For example, cross-linked neutral acrylates (e.g., poly(N-hydroxyethylmethacrylamide (pHEMA))) may be used. Any amine-reactive polymer with a molecular weight over 100,000 can serve as a substitute for poly(acrylic acid NHS ester). For example, dialdehyde dextran can be used.

The present invention also provides enhanced materials that provide combinations of variety of highly desired properties, including, but not limited to a) superabsorbability; b) a hydrophilic environment favorable for the preservation of protein activity; c) low background conveniently achieved in a single polymeric preparation; and d) gel cross-linking and surface bonding that takes place in the same reaction. For example, such properties are achieved through the use of combining appropriate components in a cross-linking reaction to produce simple chemical bonds and no fluorescent contaminants: e.g., poly(acrylic acid) and PEG diamine (polymers with superabsorbent properties); polymeric crosslinker (PEG diamine, which provides large pores); and the polymers and cross-linking chemistry (e.g., amide formation using carbodiimide and N-hydroxysuccinimide).

The present invention provides new compositions and methods for preparing three-dimensional polymeric coatings for immobilization of molecules on a surface. In preferred embodiments, the 3D coating possesses superabsorbent properties (e.g., the capability to absorb at least 100 times its weight in water) and comprises a crosslinked polyacrylic acid with carboxylic groups activated to react with primary amines of molecules to be arrayed.

In some embodiments, the present invention provides 3D networks on surfaces, methods of making such networks, and methods and composition using such modified surfaces.

The present invention is not limited by the identity of the polymer used to prepare the three-dimensional networks. Suitable polymers preferably carry no fluorescent moieties and do not form fluorescent moieties during cross-linking. In some embodiments, a fluorescent molecule or other label is added to monitor how evenly the coating was attached. Preferably when such a method is used, the label is selected so as to not interfere with a detection component (e.g., red fluorophores). This can be accomplished, for example, by adding a very low amount of a label that absorbs at an extreme end of the ultraviolet range with a low quantum yield.

In some preferred embodiments, the methods of the present invention provide cross-linked hydrogels on a surface. Cross-liking may be carried out using any suitable method, including, but not limited to, radiation-induced cross-linking; enzymatic cross-linking (e.g., transglutaminase+glutamic acid-based polyanion+diamine); and chemical cross-linking methods (e.g., polyamine+dialdehyde with reduction, polyamine+diisothiocyanate, polymaleimide+dithiol, polythioester+dithiol, polybenzotriazole carbonate+diamine, and polyvinylsulfone+dithiol/diamine). The gels may be affixed to surface using any suitable method, including, but not limited to: i) direct casting on the surface (e.g., glass surface) under a coverslip or equivalent compent to control thickness; ii) dip-coating; and iii) spin-coating.

The present invention is not limited by the pore size of the three-dimensional materials. In some preferred embodiments, the average pore size has a diameter larger than 50 nm.

The present invention is not limited by the nature of the surface onto which the three-dimensional matrix is associated. Suitable surfaces include, but are not limited to, any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). The surface may the surface of a device (e.g., medical device) or machine.

Preferred embodiments of the present invention have biological molecules such as nucleic acid molecules, proteins, lipids, carbohydrates, peptides, synthetic polymers, etc. attached to or associated with the three-dimensional matrix. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both. Preferable attachment chemistries preserve the activity of the biological molecule and do not produce background fluorescence. Suitable chemistries include, but are not limited to, amide, ester, urethane, carbonyl, thioester, disulfide, secondary amine, imine, and avidin/biotin.

### EXAMPLES

### Example 1

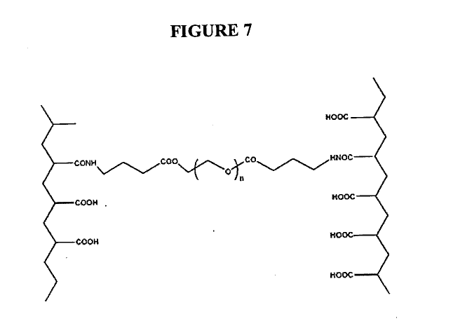

### Preparation of 3D Activated Polyacrylate-Based Gel on the Surface of Glass Slide (FIG. 7)

Poly(acrylic acid) (Mw 400 k, Aldrich, pAA) was dissolved in water at 20 mg/ml. The pH was adjusted to 6.0 with 4M NaOH. 1-ethyl-3-(3-dimethylaminopropyl) carbodiimide (20 mg/ml, EDC) and N-hydroxysuccinimide (12 mg/ml, NHS) were dissolved in pAA solution. PEG diamine (PEG-DA, Aldrich, Mw 700) was dissolved in 0.1 M MES buffer, pH 6.0 at the concentration of 20 mg/ml. This solution (0.2 ml) was mixed with 0.2 ml of activated pAA solution. At these conditions the gel is usually formed within 2 hrs. Immediately upon mixing the reaction mixture (0.2 ml) was poured on the surface of amino-modified glass slide (24×72 mm, Superamine, Telechem International, Inc.) and covered with a coverslip (24×52 mm). The slide was left in humid chamber for 16 hrs at room temperature. The coverslip was removed and the gel bonded to the slide surface was extensively washed in 5 mM EDTA solution and then in 0.1 M MES, pH 5.0. Washed surfaces were incubated with EDC (20 mg/ml) and sulfo-NHS (20 mg/ml) dissolved in 0.1 M MES, pH 5.0 for 1 hr at room temperature. The slides were washed in 0.1 MES buffer for 1 hr then in deionized water for 1 hr and then freeze-dried.

### Example 2

### Comparison of pAA Gel Surfaces with Other Commercially Available Glass Slides in a Model Binding Assay

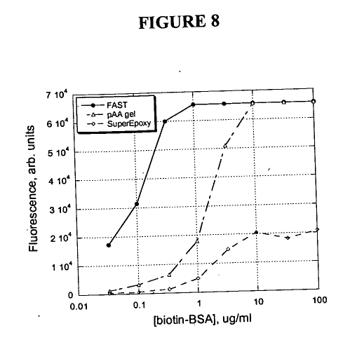

The performance of the glass slides with pAA gel surfaces were compared with other available surfaces. In particular, two other surfaces were chosen for comparison in biotinylated BSA/Alexa555-streptavidin binding assay: nitrocellulose-based FAST slide (Schleicher & Schuell, Keene, N.H.) and flat surface SuperEpoxy slide (TeleChem International, Inc. Sunnyvale, Calif.). Biotin-BSA titrations in HBS (10 mM HEPES, 150 mM NaCl; pH 7.5) were printed on the surface of all three slides starting with 100 ug/ml and allowed to incubate for 1 hr in humid chamber. Then the slides were blocked for 1 hr in StartingBlock buffer (SB, Pierce, Rockford, Ill.). Solution of Alexa555-streptavidin conjugate (1 ug/ml in SB, Molecular Probes, Inc., Eugene, Oreg.) was incubated on all surfaces under coverslip for 1 hr at room temperature. The slides were washed in HBS (3×2 min), water (1 time), dried under stream of N₂ and scanned using microarray scanner (FIG. 8).

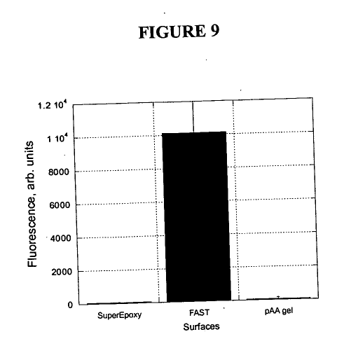

Background was measured in zero input areas on the same slide. As it can be concluded from FIG. 8, pAAgel surface has intermediate capacity between FAST and SuperEpoxy slides. However, fluorescence background on pAA gel slides is significantly (>150 times) lower as compared to FAST surface (FIG. 9) making thus pAA gel surfaces superior to both FAST and SuperEpoxy slides.

### Example 3

### Coating the Surface of Amino-Modified Glass with Agarose/Poly(Acrylic Acid) Composite Hydrogel and its Activation with 1-ethyl-3-(3-dimethylaminopropyl) carbodiimide (EDC) and N-Hydroxysuccinimide (NHS)

In this example agarose was used as an inert supporting gel material and NHS ester-activated pAA as a glass bonding and protein-binding polymer. Agarose (Gibco, 0.5 g) was melted in 200 ml of deionized water in laboratory microwave. 50 mg of pAA (mol. wt. 4000 kDa, Aldrich) as aqueous solution was added to the melt. The mixture was thoroughly mixed on a stirrer plate while the temperature was controlled using a thermometer. Initial temperature of the polymer blend was usually in a range of 65-75° C. and was allowed to decrease. EDC (30 mg) and NHS (20 mg) were added at t=45° C. Amino-modified glass slides (Superamine, Telechem International) were dipped into the activated polymer solution, incubated for 10 min and then were removed by hand or using dip coating machine. The coated slides were allowed to solidify for 30 min at room temperature in a humid chamber. Then the slides were washed in deionized water for 2 hrs with agitation. Next, the slides were activated by immersing them into the following solution: 10 mg/ml EDS, 6 mg/ml NHS in 50 mN MES, pH 5.5 for 10 min. After a brief wash in deionized water, the slides were freeze-dried.

### Example 4

### Performance Assay for Composite Hydrogel Surfaces

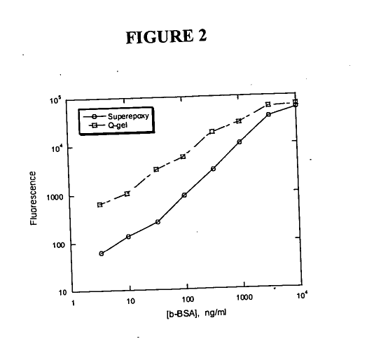

The performance of the glass slides with hydrogel surfaces were compared with Superepoxy slides (Telechem International) a typical 2D planar protein-reactive surface. A model biotin-BSA/fluorescent streptavidin binding assay was used for performance comparison. Biotin-BSA (Sigma) titrations in HBS (20 mM HEPES, 150 mM NaCl, pH 7.5) were printed in triplicates on the surface of all three slides starting with 100 ug/ml and allowed to incubate for 1 hr in humid chamber. Then the slides were blocked for 1 hr in StartingBlock buffer (SB, Pierce, Rockford, Ill.). Solution of Alexa555-streptavidin conjugate (1 ug/ml in SB, Molecular Probes, Inc., Eugene, Oreg.) was incubated on all surfaces under coverslip for 1 hr at room temperature. The slides were washed in HBS (3×2 min), water (1 time), dried under stream of N₂ and scanned using a microarray scanner (FIG. 2). FIG. 2 demonstrates that the hydrogel-coated surface demonstrated significantly higher sensitivity in biotin-BSA/fluorescent streptavidin assay, proving the superiority of 3D hydrogel surface over conventional 2D surfaces.

### Example 5

### Measurement of the Composite Hydrogel Thickness Using Confocal Fluorescence Microscopy

The slides processed as described in Example 4 were incubated in HBS and viewed using Zeiss LSM 510 confocal laser system. The fluorescence is detectable in 14 slices making the total coating thickness 13 mm.

### Example 6

### Comparative Activity of Slides

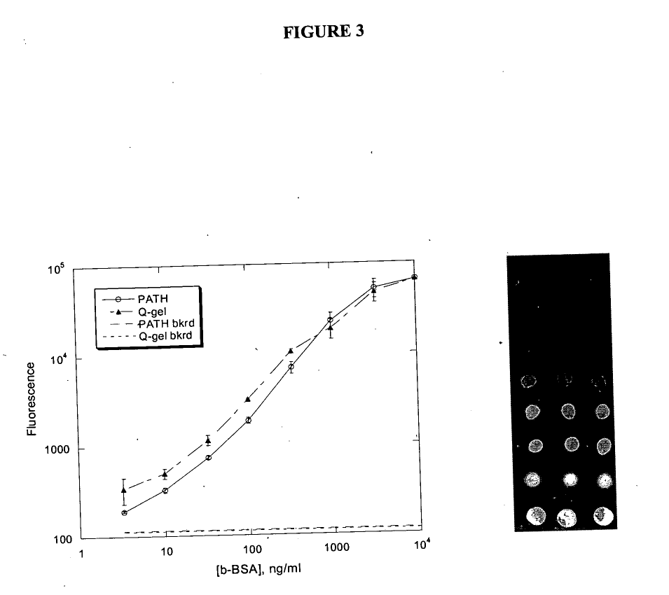

FIG. 3 shows a comparison of binding assay sensitivity between slides of the present invention (Q-gel) and available commercial slides. Dilutions of biotin-BSA in HEPES-buffered saline were spotted on a coated slide of the present invention and a popular PATH slide (GenTel Biosciences). The dilutions were developed with Alexa555-streptavidin solution. The results demonstrate superior sensitivity of coating of the present invention at lower protein concentrations. The stability of the slides of the present invention was also tested. The results indicated that protein binding ability of the surface deteriorates insignificantly even after storing the substrate for 3 months at room temperature.

### Example 7

### Preservation of Functional Activity of Membrane Proteins Immobilized on Slides

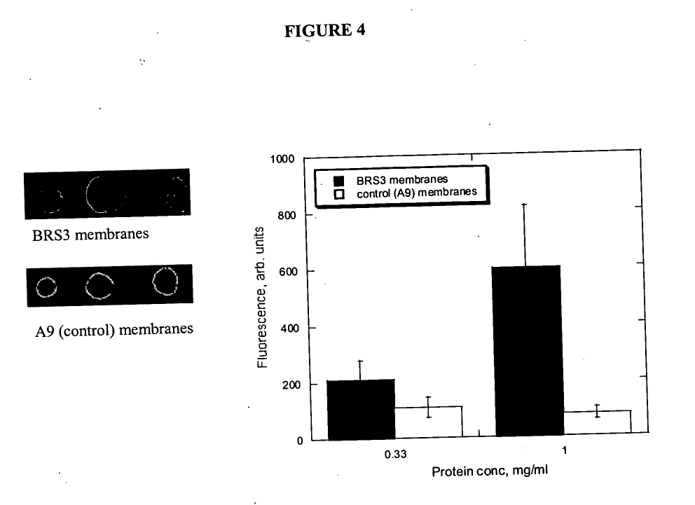

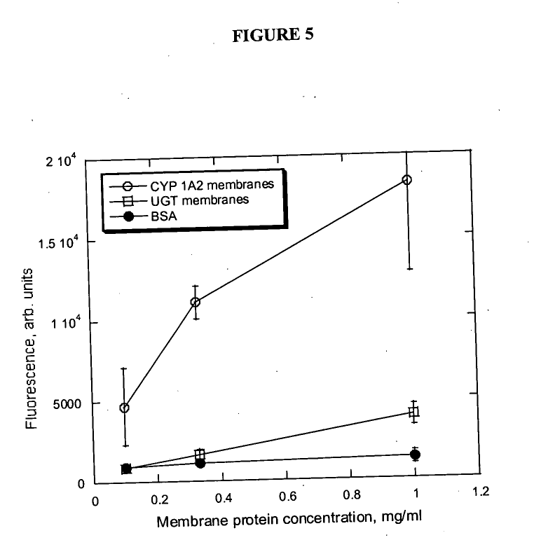

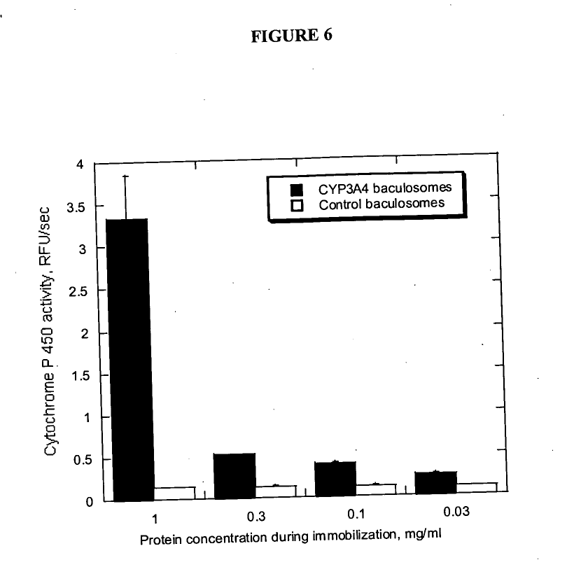

The slides of the present invention function to maintain membrane protein activity of membrane proteins immobilized on the slides. FIG. 4 shows detection of fluorescent ligand binding to membrane receptor immobilized on Q-gel surface. This figure demonstrates the binding of BODIPY-TMR bombesin (fluorescently-labeled peptide hormone) to the isolated cell membranes containing human bombesin receptor (of GPCR family). This experiment shows that the Q-gel surface is especially good for immobilization of membrane protein fractions. FIG. 5 shows detection of Cytochrome P450 1A2 (baculosomes, Invitrogen) directly immobilized on the Q-gel surface. The immobilized protein was developed using fluorescently-labeled antibodies against CYP1A2. FIG. 6 shows detection of enzyme activity of Cytochrome P450 immobilized on the Q-gel surface. For this experiment, Q-gel was immobilized on the bottom of 96-well plate. CYP3A4 baculosomes were immobilized and washed with buffer. The enzyme activity was determined using Vivid Red water-soluble substrate. This data demonstrates that membrane proteins immobilized on Q-gel surfaces retain their biological function.

All publications and patents mentioned in the above specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising a surface configured to attach a plurality of biological molecules, said surface comprising a polymeric network having an interpenetrating mesh of two or more non-covalently-linked polymers, the first polymer providing an inert hydrophilic supporting scaffold and the second polymer configured to bond said polymeric network to said surface and said biological molecules.

2. The composition of claim 1, wherein said first polymer comprises agarose.

3. The composition of claim 1, wherein said second polymer comprises poly(acrylic acid NHS ester).

4. A composition comprising a surface configured to attach a plurality of biological molecules, said surface comprising an impermeable layer and a porous matrix layer, said porous matrix layer comprising cross-linked polymers and having the properties of: i) superabsorbability; ii) hydrophilic regions; and iii) substantially no fluorescence.

5. The composition of claim 4, wherein said biological molecules are selected from the group consisting of proteins, nucleic acids, lipids, carbohydrates, peptides, and synthetic polymers.

6. The composition of claim 4, wherein said impermeable layer comprises a glass slide.

7. The composition of claim 4, wherein said cross-linked polymers comprise carboxy-containing polymer activated with water-soluble carbodiimide.

8. The composition of claim 7, wherein said carboxy-containing polymer is poly(acrylic acid).

9. The composition of claim 4, wherein said porous matrix layer is formed using a diamine crosslinker of molecular weight greater than 700.

10. The composition of claim 4, wherein said porous matrix layer is formed using a polyethylene glycol diamine crosslinker.

11. The composition of claim 10, wherein said polyethylene glycol diamine crosslinker has a molecular weight greater than 700.

12. The composition of claim 4, wherein said porous matrix layer is lyophilized.

13. A method of preparing a highly porous crosslinked polymeric network on a surface, comprising the steps of: a) providing an impermeable surface; b) associating a porous matrix layer comprising cross-linked polymers with said impermeable surface; wherein said porous matrix layer has the properties of: i) superabsorbability; ii) hydrophilic regions; and iii) substantially no fluorescence.

14. The method of claim 13, further comprising the step of attaching a biological molecule to said porous matrix layer.

15. The method of claim 14, wherein said biological molecule is selected from the group consisting of proteins, nucleic acids, lipids, carbohydrates, peptides, and synthetic polymers.

16. The method of claim 13, wherein said porous matrix layer is formed from preformed carboxy-containing polymer activated with water-soluble carbodiimide.

17. The method of claim 13, wherein the porous matrix layer is associated with the impermeable layer by direct casting.

18. The method of claim 13, wherein the porous matrix layer is associated with the impermeable layer by dip-coating.

19. The method of claim 13, wherein the porous matrix layer is associated with the impermeable layer by spin-coating.
